(19) United States
(12) Patent Application Publication
(10) Pub. No.: US 2022/0125870 A1
(43) Pub. Date: Apr. 28, 2022

(54) METHOD FOR PRODUCING PLANT MATERIALS HAVING REDUCED VARIANCE

(71) Applicant: Bionorica SE, Neumarkt (DE)

(72) Inventors: Michael POPP, Neumarkt (DE); Stefanie DELUEG, Innsbruck (AT); Daniel INTELMANN, Munich (DE); Stefan SCHONBICHLER, Innsbruck (AT); Martin DITTMER, Absam (AT); Moritz RUBNER, Pielenhofen (DE)

(21) Appl. No.: 17/427,382

(22) PCT Filed: Jan. 31, 2020
(86) PCT No.: PCT/EP2020/052430

§ 371 Date: Jul. 30, 2021

(30) Foreign Application Priority Data
Jan. 31, 2019 (EP) 19154942.7

(51) Int. Cl. A61K 36/70; A61K 36/85

(52) U.S. Cl. CPC A61K 36/70; G01N 30/7206; A61K 2236/10; A61K 36/85

(57) ABSTRACT

The invention relates to a method for producing standardised or quantified plant materials having reduced variance in phytonutrients, in particular from medicinal plants, using a mixture calculator.

The invention relates to a method for producing standardised or quantified plant materials having reduced variance in phytonutrients, in particular from medicinal plants, using a mixture calculator.

Medicinal plants contain ingredients with pharmacological effects, possibly enriched in certain parts of the plant such as roots, leaves, flowers or fruits, and form the basis for a considerable number of medicinal products. There are various methods for obtaining these ingredients, most of which work according to the principle of extraction of some kind, including maceration or percolation of the plants with a suitable extraction agent or solvent, and result in a more or less selective solution and enrichment of certain active plant substances or groups of active substances in the extraction agent or extract. The extracts may be liquid, semi-solid or solid and, in particular, a dry extract (Extracta sicca), in which case, for example, the extraction residue, the fluid extract obtained (Extracta fluida) or the tincture obtained (Tincturae) is concentrated to dryness. Drying can be carried out by means of fluidised bed drying or a concentration to a thick or spissum extract (viscous extract, Extracta spissa) with subsequent vacuum belt drying or tray drying, see also for example EP 0 753 306 B1 in the name of the applicant.

Diagram:

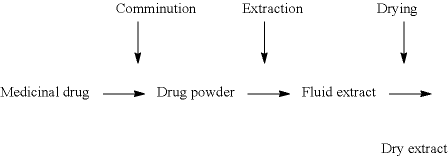

The applicant produces and sells, for example, such extracts from medicinal plants as Bronchipret®, Sinupret Extract®, Canephron®, Imupret®, etc. as well as on a drug basis (Sinupret®).

The extracts in question are registered medicinal products or authorised medicinal products, so-called traditional or rational phytopharmaceuticals (incl. well established medicinal use), which require an adequate dosage as well as a prescription appropriate to the indication, taking into account the risk-benefit ratio.

Monographs (also: pharmacopoeial regulations (German Pharmacopoeia (DAB), European Pharmacopoeia (EuAB or Ph.Eur. for Pharmacopoea Europaea)) describe plants for provision as medicinal products and their preparation, in particular with regard to their efficacy-determining constituents, effects, indications for use, contraindications, side effects, interactions, dosage and dosage form. Positively monographed are plants for which Commission E (Licensing and Preparation Commission at the BfArM, Germany (Federal Institute for Drugs and Medical Devices)) or the “Committee on Herbal Medicinal Products (HMPC)” at the EMA could list sufficient proof of efficacy and safety on the basis of the available studies.

According to Ph.Eur. and for the purposes of the present invention, extracts are preparations of liquid, semi-solid or solid nature prepared from, usually dried, herbal drugs.

So-called standardised extracts are adjusted within permissible limits to a given content of efficacy-determining ingredients (lead substances). The adjustment can be made by mixing batches of extracts and/or by adding excipients.

So-called quantified extracts are adjusted to a defined range of efficacy-determining ingredients (lead substances). The adjustment can be made by mixing batches of extracts.

Extracts or batches may be mixed by a mixing device, using a mixture calculator that adjusts the mixing ratio of the extracts with the help of a computer-aided calculator.

However, plant extracts or drug batches consist of a variety of phytonutrients. The content of such phytonutrients may vary greatly depending on various parameters, such as the growing region, weather conditions, care, harvesting methods, time of harvest, and many more.

Consequently, plant extracts or drug batches contain different contents of phytonutrients depending on the origin, history, harvest time, production process and other parameters, which may lead to qualitative differences in the individual constituents or in a batch from a totality of processed individual constituents as well as between batches themselves.

Therefore, plant extracts as well as powdered drugs have the problem of phytochemical batch variability. In order to be able to guarantee a consistent therapeutic success in a treatment with a phytopharmaceutical, the composition of the plant-based ingredient of an extract or phytopharmaceutical must remain as identical or stable as possible from batch to batch, i.e. there is a need for low batch variability.

This is ultimately due to the fact that the phytonutrients of the extracts or batches exhibit a natural, biological variance (“biological space”).

Phytonutrients are in particular secondary plant substances. Secondary plant substances, or secondary metabolites, are derived from products of anabolic and catabolic metabolism, especially carbohydrates and amino acids. For medicinal plants, the secondary plant substances are decisive for their suitability as active substances. Secondary plant substances include, in particular, phenolic, isoprenoid and alkaloid compounds such as phenols, polyphenols, flavonoids, caffeic acid derivatives, xanthones, terpenes, steroids and other natural substances. Secondary plant substances in particular may vary greatly in their occurrence and content in individual constituents or batches.

Phytonutrients in plant materials can be characterised qualitatively and quantitatively by analytical methods (e.g. chemotaxonomy). Primarily, gas and/or liquid chromatography are coupled with a mass spectrometer, such as GC-MS or LC-MS. It is common to display signals (peaks [m/z]) as a function of the retention time (chromatogram). This also allows the determination of contents (e.g. w/w or v/v) of phytonutrients in the drug or in the extract or in a plant material via integrated peak areas obtained.

No techniques are described in the prior art that allow the production of plant extracts or drug batches or plant materials while achieving a reduction in the variance in phytonutrient contents.

Therefore, it is an object of the present invention to provide plant material(s) having a reduced variance (variability) in phytonutrient content in a plant material, in particular having a reduced batch variability.

The provision of plant material(s) with reduced variance (synonym: variability) in phytonutrient content leads to optimised batch homogeneity. An optimised batch advantageously allows an improved reproducibility of the batches, and consequently a new quality standard is guaranteed. This is also of great importance for approval issues. For example, the FDA (US) has not yet granted approval for a plant extract or powdered drugs as a multi-substance mixture. Furthermore, a lower error tolerance in comparative studies is made possible, which in turn improves the study quality and is comparable with synthetic medicaments.

Therefore, in order to solve this problem, the invention relates to a method for producing plant material having reduced variance in phytonutrient content, wherein at least one variance marker is used and at least two batches are mixed by means of a mixture calculator.

In a preferred embodiment, the object is achieved by a method according to the invention for producing plant material having reduced variance in phytonutrient content, said method having the following steps (see also Example B):
i.) determining signal intensities for phytonutrients in two or more batches by means of a detector, in particular GC-MS and/or LC-MS,
ii.) identifying at least one phytonutrient which makes a contribution, preferably the greatest contribution, to the variance and identifying its determination of the natural span (referred to hereinbefore and hereinafter as a variance marker),
iii.) setting one or more limit values which is/are smaller than the natural span from ii.),
iv.) mixing at least two batches, wherein at least one limit value from iii.) is taken into consideration by means of a mixture calculator,
v.) optionally, repeating steps i.) to iv.).

In step ii.) a determination of the variance in the phytonutrient content can be carried out.

The method according to the invention therefore advantageously allows the production of stable or identical, homogeneous batches, so that in particular the reproducibility of the batches is guaranteed. This is due in particular to the fact that the variance in the phytonutrients is reduced via the identified at least one variance marker, which allows a maximum reduction of the total variance in the phytonutrients. This variance marker identified in accordance with the invention contributes significantly to the spread and is reduced in its effect on the overall variability by the method according to the invention.

The term “phytonutrient content” means the relative or absolute amount (mass, weight) of one or more phytonutrient(s) or its/their relative or absolute concentration(s) (w/w) (v/v).

In a further preferred embodiment, the method according to the invention for producing plant material having reduced variance in phytonutrient content comprises the following steps (see also Example C):
i.) determining signal intensities for phytonutrients in two or more batches by means of a detector, in particular GC-MS and/or LC-MS,
ii.) dividing the signals into two or more sub-ranges, and summing the signal intensities of the phytonutrients within each sub-range,
iii.) identifying at least one sub-range which makes a contribution, preferably the greatest contribution, to the variance and identifying its determination of the natural span (referred to hereinbefore and hereinafter as a variance marker),
iv.) setting one or more limit values which is/are smaller than the natural span from iii.),
v.) mixing at least two batches, wherein at least one limit value from iv.) is taken into consideration by means of a mixture calculator,
vi.) optionally, repeating steps i.) to v.).

Within the scope of step iii.), a determination of the variance in the phytonutrient content can be carried out.

This embodiment particularly advantageously permits the provision of plant materials from complex plant materials which have a large number of phytonutrients, in particular more than 300 phytonutrients, in particular secondary plant substances. The sub-ranges according to the invention allow a systematic representation of the complex signal intensities with summation of those signal intensities.

However, according to the invention, the sub-ranges can be broad as well as very narrow (focused), i.e. each sub-range can contain, for example, only one signal of a phytonutrient. Consequently, this embodiment may also comprise the first embodiment of the method (supra).

The invention is explained in more detail below.

The expression “determining signal intensities for phytonutrients in two or more batches by means of a detector, in particular GC-MS and/or LC-MS” preferably includes the use of liquid chromatography (LC), preferably HPLC, in conjunction with high-resolution mass spectrometry, such as time-of-flight (TOF) devices, in particular high-resolution HPLC-TOF-MS. The aforementioned terms GC-MS and/or LC-MS are not to be understood restrictively, and include any embodiment of a device suitable for this purpose. In particular, any detectors can be used, such as UV-VIS, thermal conductivity detector (WLD), flame ionisation detector (FID), etc. It is only necessary that the plant materials used in the course of the determination by means of a detector, in particular GC-MS and/or LC-MS, may present corresponding signals (peaks [m/z]) or signal intensities as a function of the retention time, a so-called chromatogram.

Therefore, such detectors are also included in accordance with the invention which present signal intensities as a function of the retention time in a chromatogram.

If necessary, the variance markers according to the invention identified by preferably GC-MS and/or LC-MS or correspondingly represented in the sub-ranges according to the invention can be supplemented by further quality-determining analytical methods (for example: IR, NIR, Raman spectroscopy, atomic absorption spectroscopy, wet chemical assays (e.g. polyphenol determination according to Folin-Ciocalteu), fragmenting mass spectrometry techniques (MS″)).

It is preferred that at least 100, 200 or 300 signals (or signal intensities) or more are determined in at least one batch. Furthermore, it is preferred that the signal intensities are determined in five batches, preferably different from each other, in particular ten batches and more.

In a further preferred embodiment, the signals or signal intensities for the particular batch of a plant extract can be recorded in a database or memory.

The expression “determining the variance in the phytonutrient content” can be carried out as follows, with the determined signals or signal intensities for a batch or n batches being arranged in a matrix:

$$A = ( \begin{matrix} & {Signal}_{1} & {Signal}_{2} & ⋯ & & {Signal}_{k} \\ {Batch}_{1} & {a}_{1 , 1} & ⋯ & ⋯ & & {a}_{1 , k} \\ {Batch}_{2} & {a}_{2 , 2} & ⋱ & ⋯ & & {a}_{2 , k} \\ ⋮ & ⋮ & ⋯ & ⋱ & ⋮ & \\ {Batch}_{n} & {a}_{n , 1} & ⋯ & ⋯ & & {a}_{n , k}\end{matrix} )$$

From each column, the mean values x̅ᵢ and the standard deviations sᵢ can be calculated. The mean relative standard deviation (in %) results in

$$RSDX = \frac{{\sum }_{i = 1}^{k} {s}_{i}}{{\sum }_{i = 1}^{k} \overline{{x}_{i}}}$$

According to the invention, the mean relative standard deviation can be determined mathematically from the aforementioned matrix and consequently used to determine the variance reduction. This determination may be performed by a calculator, computer-aided.

The expression “identifying at least one phytonutrient that makes a contribution, preferably the greatest contribution, to the variance and identifying its determination of the natural span (variance marker)” or “identifying at least one sub-range that makes a contribution, preferably the greatest contribution, to the variance and identifying its determination of the natural span (variance marker)” may be performed as follows:

For the determination of the variance markers, all mathematical methods may be used that are able to identify variables with the greatest variance from a data matrix. For example, in the simplest form, the variances of all variables may be calculated and then the variables with the greatest variance may be selected. Preferably, however, a principal component analysis (PCA) should be used for the analysis. In PCA, a so-called “score plot” (FIG. 1) is generated on the basis of the data obtained, in which the group of batches used is shown in a diagram. The more widely the points of a group (or, as in FIG. 4, of several groups, calculated within a PCA) within the score plot are distributed over the coordinate system and the larger the surrounding confidence ellipse, the greater the variance underlying the group. On the other hand, PCA also generates a “loading plot” (see FIG. 2), from which it can be derived which signals make a contribution, preferably the greatest contribution, to the total variance and, as applicable, also correlate with other signals. The variance markers are selected in such a way that the loading value of each signal intensity sum of the sub-ranges over in this case 5 principal components (see also Example D), explanation of >80% total variance) is first absolutised, then summed, and lastly the loading sums are sorted in descending order. To make the selected signals even more representative of the total extract, a high correlation with as many other signals as possible can be used as an additional criterion. The loading sums with the highest resulting values contain those sub-ranges (and thus signal candidates) that may be used as variance markers. FIG. 3 shows that there are usually a limited number of sub-ranges (signals) that show a high variance.

It is then expedient to determine the absolute content of the signals obtained for the variance markers found (via LC-MS, LC-DAD, NIR or other analytical methods) in order to obtain comparable data from several batches, preferably measured over longer periods of time.

The PCA used according to the invention is, in the sense of the present invention, a mathematical method to extract relevant information from a very comprehensive and complex data set and to separate the statistical noise. This is a so-called “data mining” technique, which simplifies a data set while still retaining as much information content as possible.

The result of a PCA is usually constituted by two blocks of information: A so-called “score plot” and a “loading plot” linked to it. In the score plot, the samples are generally grouped according to their “properties” (according to the invention, the properties are the intensity values of the measured (LC/MS) signals). Samples that are very similar in all their properties are grouped closely together; those that are more different are grouped further apart. The relative extent of the point cloud of samples in the score plot (for example, when unmixed samples are compared with mixed samples) also tells us something about their underlying variability. A compact point cloud has less inherent variance than an extended point cloud.

The loading plot, on the other hand, shows which properties (or here: LC/MS signals) are significantly responsible for the positioning of the objects in the score plot. Signals that are far away from the coordinate origin in the loading plot contribute strongly to a positioning (and thus have a great influence on the variability of the samples) and are therefore a promising optimisation criterion in the context of the mixture calculation.

The expression “setting one or more limit values smaller than the natural span” means setting a limit value or range of contents smaller than the natural span of the at least one variance marker. The value 0 is included here. Once the variance markers have been determined, their natural span may be determined from the determined signal intensities.

The expression “mixing at least two batches, taking into account at least one set limit value by means of a mixture calculator” means that an algorithm is provided which calculates, by means of a computer-aided mixture calculator, the ratio in which at least two or more batches must be mixed so that at least one variance marker lies within the newly selected, limited interval or (sub-)range.

For the calculation, a linear system of inequalities is solved taking into account constraints (e.g. Lay, David C. (Aug. 22, 2005), Linear Algebra and Its Applications (3rd ed.), Addison Wesley). If n batches are available in the mixing pool and k limit value intervals are taken into account, this may be written as G*\vec{x}≥\vec{h} (FN 1). G is here a matrix with 2*n rows and k columns. Each row contains the values of the measured individual parameters to be optimised for each batch, wherein the rows 1 . . . n are given a positive sign and the rows (n+1) . . . 2*n a negative sign. The vector \vec{h} contains the limits of the mixing intervals, wherein the entry h₁ . . . h_{k/2} contains the lower limit values and h_{k/2+1} . . . hₖ contains the upper limit values—these must also be given a negative sign. When solving this system of inequalities, a matrix with constraints must also be taken into account and solved at the same time. In this, the calculation of the percentage shares, as well as the use of individual batches, as applicable, is enforced. It is formulated as A*\vec{x}=\vec{b}, wherein A is a matrix with m rows (with m>1) and k columns. The vector \vec{b} also has m entries, which always have the value 1. The first row of A also has only 1 as an entry; in the other rows, however, it may additionally be defined

¹In the following, matrices are always written in bold (e.g. A), vectors with an arrow (e.g. \vec{x}) and scalars in italics (e.g. n)).

whether a deliberately selected mixture share of some batches is to be taken into account, or whether individual batches are to be deliberately excluded from consideration.

The mixing problem formulated in this way is solved using an appropriate linear optimisation algorithm, and the ratios to be mixed are specified.

In a further preferred embodiment, the variance from the data sets is preferably presented using a Principal Component Analysis (PCA). In particular, in a further preferred embodiment, the PCA may be obtained from the signal intensity sums of the sub-ranges of a mass defect plot. In a mass defect plot, the determined signals are plotted as m/z against the mass defect. The mass defect is calculated by dividing the decimal places of the measured mass by the total mass of the measured mass (see also Example C). Phytonutrients with similar mass and also similar atomic composition are found close to each other in the plot (see FIG. 11: Mass defect plot). According to the preferred embodiment already explained, the sub-ranges may now be shown as sub-areas. It is particularly advantageous that, in the course of the presentation via the mass defect plot, such sub-areas may represent secondary plant substances such as phenols, flavonoids and many others, and a variance marker for this sub-range or sub-area may be easily identified.

In the context of the present invention, a batch, in particular a plant material batch, such as a drug or extract batch, is understood to be the totality of units produced in a batch process which results in the production of delimited substance quantities by subjecting quantities of feedstock to an ordered sequence of process activities using one or more pieces of equipment within a limited period of time. The starting product is usually the plant drug from which the plant extract or the processed plant drug is obtained.

According to the invention, two or more batches may be mixed in a mixer in accordance with the invention, the allocation of the individual batches into a mixture being predetermined by the mixture calculator, which in turn may be programmed by an algorithm which in particular takes into account the limit value according to the invention for a variance marker in accordance with the method according to the invention.

In the context of this invention, plant materials (singular or plural) include any plant material, for example plant parts, such as leaves, stems, roots, flowers. In particular, plant materials may be in the form of their plant drugs as well as plant extracts (supra).

Furthermore, the invention comprises the obtained plant materials, in particular plant extracts, which may be obtained by the method according to the invention. The plant materials obtained in accordance with the invention, in particular plant extracts, have at least a modified phytonutrient content, in particular these obtained plant materials, in particular plant extracts, have a reduced variance in phytonutrient content compared to the starting plant material. The obtained plant materials, in particular plant extracts, are specific in respect of the reduced variance, and at least one variance marker has an altered phytonutrient content. In addition, the spread of the obtained phytonutrient content is reduced.

Therefore, the invention relates to a plant material with reduced variance in phytonutrient content, which is obtained or produced or obtainable by the methods according to the invention.

In the context of the present invention, a “plant extract” as well as “plant material” is understood to be a multi-component mixture of natural substances which contains more than two natural substances, in particular more than 10 or 100 natural substances, in particular more than 200, 300, 500 or 1,000 natural substances. Plant extracts may be obtained from plant materials, for example, by means of extraction, percolation or maceration. Solvents such as water, C1-C5 alcohols, ethanol, or other solvents with sufficient polarity may be used as extraction agents. A common extraction is, for example, a mixture of water/ethanol (50:50, 70:30, 30:70).

The following genera, in particular medicinal plants, are preferred in accordance with the invention for plant extracts and plant materials:

Equiseti, Juglandis, Millefolii, Quercus, Taraxaci, Althaeae, Matricariae, Centaurium, Levisticum, Rosmarinus, Angelica, Artemisia, Astragalus, Leonurus, Salvia, Saposhnikovia, Scutellaria, Siegesbeckia, Armoracia, Capsicum, Cistus, Echinacea, Galphimia, Hedera, Melia, Olea, Pelargonium, Phytolacca, Primula, Salix, Thymus, Vitex, Vitis, Rumicis, Verbena, Sambucus, Gentiana, Cannabis, Silybum.

The following species, in particular medicinal plants, are preferred in accordance with the invention for plant extracts and plant materials:

Equiseti herba (horsetail herb), Juglandis folium (walnut leaves), Millefolii herba (yarrow herb), Quercus cortex (oak bark), Taraxaci herba (dandelion herb), Althaeae radix (marshmallow root) and Matricariae flos (resp. Flos chamomillae (camomile flowers)), Centaurium erythraea (centaury), Levisticum officinale (lovage), Rosmarinus officinalis (rosemary), Angelica dahurica (Siberian angelica, PinYin name: Baizhi), Angelica sinensis (Chinese angelica, PinYin name: Danggui), Artemisia scoparia (broom mugwort, PinYin name: Yinchen), Astragalus membranaceus (var. Mongolicus) (tragacanth root, Chin.: Huang-Qi), Leonurus japonicus (lion's ear, Chin.: T'uei), Salvia miltiorrhiza (red root sage, Chin.: Danshen), Saposhnikovia divaricata (Siler, PinYin name: Fangfeng), Scutellaria baicalensis (Baikal hellebore, Banzhilian), Siegesbeckia pubescens (Heavenly herb, PinYin name: Xixianciao), Armoracia rusticana (Horseradish), Capsicum sp. (capsicum), Cistus incanus (cistus), Echinacea angustifolia (coneflower), Echinacea purpurea (coneflower), Galphimia glauca, Hedera helix (ivy), Melia toosendan (Chinese elder fruit, Chin.: Chuan Lian Zi), Olea europaea (olive), Pelargonium sp. (pelagornia), Phytolacca americana (pokeweed), Primula veris (cowslip), Salix sp. (willow), Thymus L. (thyme), Vitex agnus castus (monk's pepper), Vitis vinifera (noble vine), Rumicis herba (sorrel herb), Verbena officinalis (verbena), Sambucus nigra (black elder), Gentiana lutea (yellow gentian), Cannabis sativa (hemp), Silybum marianum (milk thistle).

Also included in accordance with the invention are mixtures of the above genera and/or species.

### EXAMPLES AND FIGURES

The following examples serve to explain the invention in greater detail without, however, limiting the invention to these examples.

FIGS. 1-12 are explained above and below.

FIG. 13 summarises the possibilities for determining the variance markers listed in Examples B, C and D. These embodiments are not to be read exhaustively.

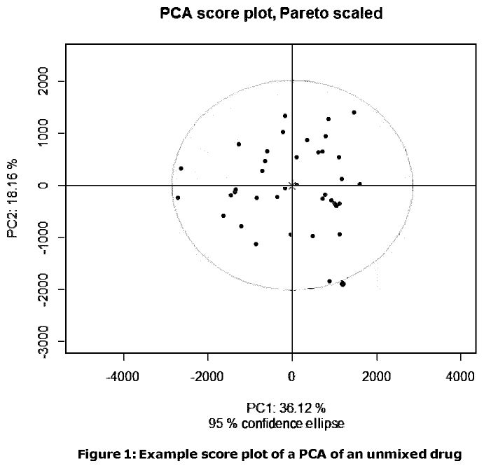

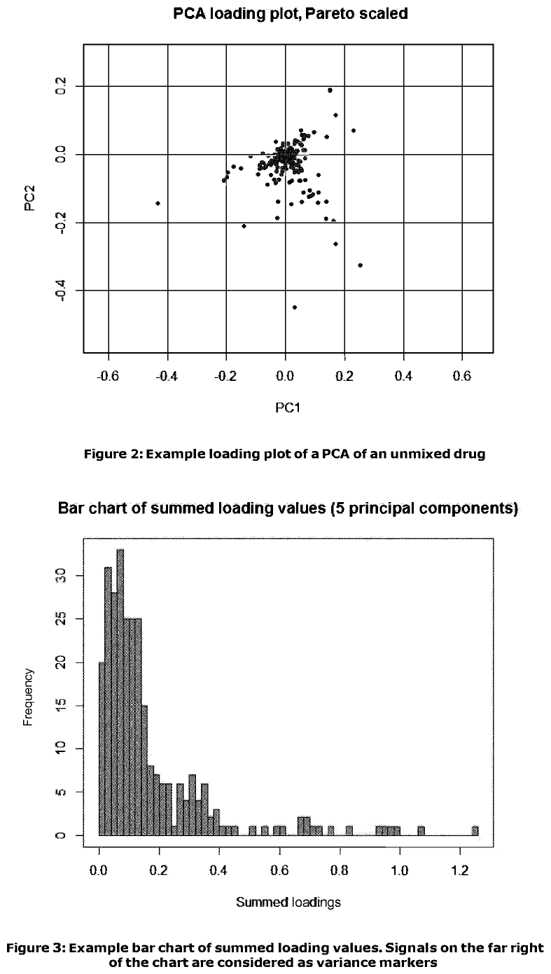

### Example A

### Examples of Mixing Success

Primula veris

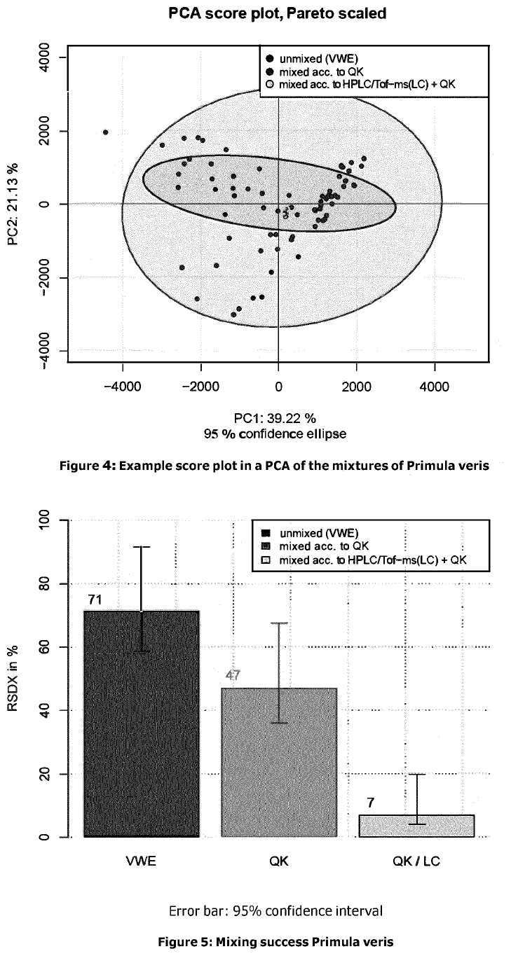

For the drug Primula veris, 30 unmixed batches were first measured by HPLC/ToF-MS, their natural variance (i.e. the RSDX (supra)) was determined, and then the variance markers were determined according to the method described above. With this information, a mixture calculation was then carried out for several batches, and these were produced in the laboratory and were measured again by means of HPLC/ToF-MS. For the mixture calculation and subsequent measurement, 5 variance markers with the highest absolute summed loading values were used. A comparison of the RSDX values obtainable in this way is shown in FIG. 5. For the example of Primula veris, a PCA was also calculated with data of mixed and unmixed samples and a confidence ellipse was placed around each sample group (FIG. 4). The smaller the area of this ellipse, the lower the variance, which here, as expected, is lowest in the mixed samples. This visualises the same information in an alternative way to the RSDX (supra).

Rumex crispus

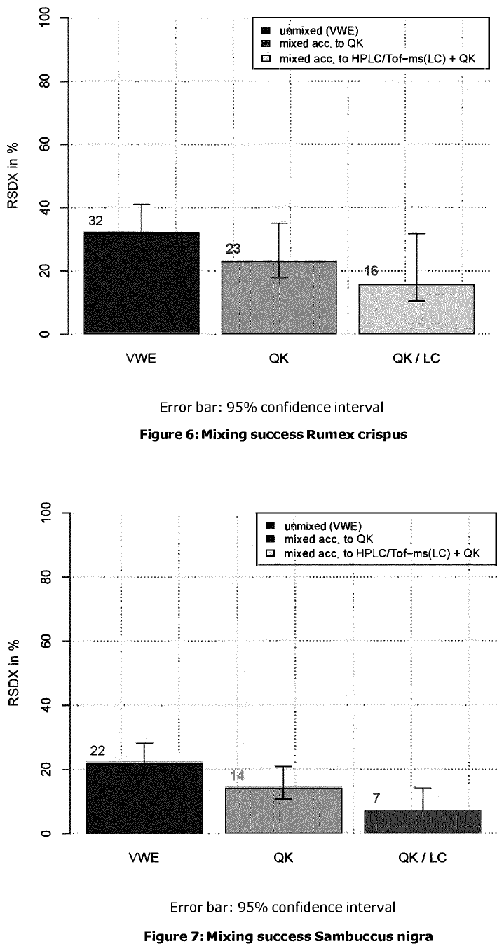

For the drug Rumex crispus, 40 unmixed batches were also measured first, and then the same procedure as for Primula veris was followed. A comparison of the RSDX values obtained is shown in FIG. 6.

Sambuccus nigra

For the drug Sambuccus nigra, 40 unmixed batches were also measured first, and then the same procedure as for Primula veris was followed. A comparison of the RSDX values obtained is shown in FIG. 7.

Verbena officinalis

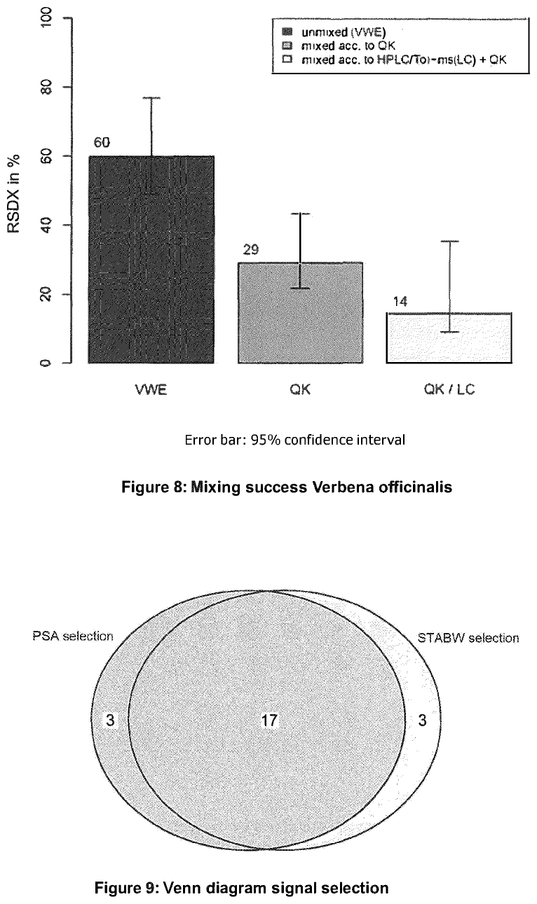

For the drug Verbena officinalis, 30 unmixed batches were also measured first, and then the same procedure as for Primula veris was followed. A comparison of the RSDX values obtained is shown in FIG. 8.

### Example B

Example of the Procedure for Variance Reduction of Plant Materials by Mixture Calculation in Rumex crispus

Extracts of the original data sets are shown, which sufficiently convey the procedure according to the invention.

i) Determination of Signals of Phytonutrients by LC/MS for Several Batches

The result is typically a table like the following—the numbers are the measured intensity values from an LC/MS measurement, for example.

| | Signal | Signal | Signal | Signal | Signal | Signal | Signal | Signal | Signal | Signal | Signal | Signal |
|---|---|---|---|---|---|---|---|---|---|---|---|---|
| | 1 | 2 | 3 | 4 | 5 | 6 | 7 | 8 | 9 | 10 | 11 | 12 |
| Batch_1 | 16711 | 5763 | 14366 | 8912 | 8521 | 24000 | 29252 | 11208 | 9471 | 8302 | 7189 | 5317 |
| Batch_2 | 16797 | 5961 | 14480 | 8713 | 9170 | 23294 | 25059 | 5834 | 7969 | 8250 | 5102 | 3937 |
| Batch_3 | 16411 | 6182 | 14636 | 8096 | 7357 | 23788 | 21161 | 7111 | 9148 | 7458 | 4075 | 5450 |
| Batch_4 | 16800 | 7030 | 12939 | 8800 | 8293 | 24433 | 19279 | 6319 | 9599 | 8005 | 4291 | 6829 |
| Batch_5 | 16677 | 6739 | 13440 | 9141 | 9954 | 23389 | 20415 | 6159 | 10361 | 8324 | 2171 | 6645 |

ii) Determination of the Total Variance/Total Standard Deviation of the 5 Samples

| | Signal | Signal | Signal | Signal | Signal | Signal | Signal | Signal | Signal | Signal | Signal | Signal |
|---|---|---|---|---|---|---|---|---|---|---|---|---|
| | 1 | 2 | 3 | 4 | 5 | 6 | 7 | 8 | 9 | 10 | 11 | 12 |
| Mean value | 16679 | 6335 | 13972 | 8733 | 8659 | 23781 | 23033 | 7326 | 9310 | 8068 | 4566 | 5636 |
| Standard deviation | 159 | 533 | 742 | 390 | 973 | 465 | 4100 | 2220 | 872 | 364 | 1818 | 1169 |
| Rel. standard deviation | 0.95 | 8.41 | 5.31 | 4.47 | 11.23 | 1.95 | 17.80 | 30.31 | 9.36 | 4.51 | 39.81 | 20.74 |
| (in %) | | | | | | | | | | | | |

| | |
|---|---|
| Sum of the mean values | 136097 |
| Sum of the standard deviations | 13804 |
| Mean relative standard deviation (RSDX) in % | 10.14 |

iii) Identification of the Signals that Contribute Most to the Variance/Standard Deviation

Calculation of the Loadings of the Table from Step i) by Means of a Principal Component Analysis (PCA) (e.g. 5 Principal Components):

| | PC1 | PC2 | PC3 | PC4 | PC5 |
|---|---|---|---|---|---|
| Signal1 | 0.109279 | 0.412709 | −0.046291 | −0.523313 | −0.689587 |
| Signal2 | 0.429376 | −0.111223 | −0.019776 | −0.179957 | 0.030141 |
| Signal3 | −0.416814 | −0.070444 | 0.164048 | 0.221163 | −0.299226 |
| Signal4 | 0.214981 | 0.454797 | −0.077392 | 0.11073 | 0.361717 |
| Signal5 | 0.182568 | 0.418618 | 0.271278 | 0.18247 | 0.151153 |
| Signal6 | 0.090994 | −0.153925 | −0.571312 | −0.340579 | 0.308789 |
| Signal7 | −0.350032 | 0.291495 | −0.176306 | 0.116159 | 0.05639 |
| Signal8 | −0.231543 | 0.129218 | −0.49517 | 0.318806 | −0.107474 |
| Signal9 | 0.313837 | −0.004629 | −0.276986 | 0.533461 | −0.322529 |
| Signal10 | 0.070295 | 0.52119 | 0.004128 | 0.023006 | 0.098764 |
| Signal11 | −0.33773 | 0.15198 | −0.360046 | −0.220731 | 0.050834 |
| Signal12 | 0.385359 | −0.100694 | −0.28623 | 0.185295 | −0.232151 |

Sum of the Absolute Values for all Signals:

| Signal1 | Signal2 | Signal3 | Signal4 | Signal5 | Signal6 | Signal7 |
|---|---|---|---|---|---|---|
| 1.781179 | 0.770473 | 1.1716956 | 1.2196165 | 1.2060872 | 1.465598 | 0.990382 |

| Signal8 | Signal9 | Signal10 | Signal11 | Signal12 |
|---|---|---|---|---|
| 1.2822101 | 1.451442 | 0.7173837 | 1.1213197 | 1.1897284 |

Sorting from the Largest to the Smallest Value—Identification of the 5 Most Important Signals for Later Optimisation (Here Highlighted in Orange):

| Signal1 | Signal6 | Signal9 | Signal8 | Signal4 | Signal5 | Signal12 |
|---|---|---|---|---|---|---|
| 1.781179 | 1.4655984 | 1.4514416 | 1.2822101 | 1.2196165 | 1.206087 | 1.1897284 |

| Signal3 | Signal11 | Signal7 | Signal2 | Signal10 |
|---|---|---|---|---|
| 1.1716956 | 1.1213197 | 0.9903817 | 0.7704728 | 0.7173837 |

Determination of the Natural Spans of the 5 Identified Signals:

| | Signal1 | Signal6 | Signal9 | Signal8 | Signal4 |
|---|---|---|---|---|---|
| Range Minimum | 16411 | 23294 | 7969 | 5834 | 8096 |
| Mean value | 16679 | 23781 | 9310 | 7326 | 8733 |
| Range Maximum | 16800 | 24433 | 10361 | 11208 | 9141 |

iv) Determination of the Phytonutrient Content Underlying the Variance Markers

This may be performed optionally, so that specific contents (for example in mg/L or the like) are assigned to the intensities. For this purpose, corresponding quantification methods are available for various analytical methods. However, this step may also be skipped (as in this example), as this only represents a linear transformation of the intensities.

v) Setting a New, Reduced Span

The allowed minimum is raised, the allowed maximum is lowered, thus reducing the allowed span of the mixture(s) (see FIG. 14):

| | Signal1 | Signal6 | Signal9 | Signal8 | Signal4 |
|---|---|---|---|---|---|
| New permitted | 16545 | 23537 | 8639 | 6580 | 8414 |
| minimum | | | | | |
| Mean value | 16679 | 23781 | 9310 | 7326 | 8733 |
| New permitted | 16740 | 24107 | 9835 | 9267 | 8937 |
| maximum | | | | | |

vi) Performance of a Mixture Calculation

After the calculation, the following handling instruction results for the batches to be used for mixing:

| | Share in % |
|---|---|
| Batch 1 | 59.34 |
| Batch 2 | 35.53 |
| Batch 5 | 5.13 |

Expected Result for this Mixture Example:

| | Signal 1 | Signal 6 | Signal 9 | Signal 8 | Signal 4 |
|---|---|---|---|---|---|
| Expected signal | 16740 | 23718 | 8983 | 9040 | 8853 |
| intensities | | | | | |

All expected signal intensities are within the desired reduced span.

vii) This Calculation May be Repeated Accordingly, for Example, Remixing Other Batches.

### Example C

Example of the Procedure for Variance Reduction of Plant Raw Materials by Mixture Calculation in Rumex crispus

Extracts of the original data sets are shown, which sufficiently convey the procedure according to the invention.

i) Determination of Signals of Phytonutrients by LC/MS for Several Batches

The result is typically a table like the following—the numbers are the measured intensity values from, for example, an LC/MS measurement.

| | Signal | Signal | Signal | Signal | Signal | Signal | Signal | Signal | Signal | Signal | Signal | Signal |
|---|---|---|---|---|---|---|---|---|---|---|---|---|
| | 1 | 2 | 3 | 4 | 5 | 6 | 7 | 8 | 9 | 10 | 11 | 12 |
| Batch_1 | 16711 | 5763 | 14366 | 8912 | 8521 | 24000 | 29252 | 11208 | 9471 | 8302 | 7189 | 5317 |
| Batch_2 | 16797 | 5961 | 14480 | 8713 | 9170 | 23294 | 25059 | 5834 | 7969 | 8250 | 5102 | 3937 |
| Batch_3 | 16411 | 6182 | 14636 | 8096 | 7357 | 23788 | 21161 | 7111 | 9148 | 7458 | 4075 | 5450 |
| Batch_4 | 16800 | 7030 | 12939 | 8800 | 8293 | 24433 | 19279 | 6319 | 9599 | 8005 | 4291 | 6829 |
| Batch_5 | 16677 | 6739 | 13440 | 9141 | 9954 | 23389 | 20415 | 6159 | 10361 | 8324 | 2171 | 6645 |

ii) Division of the Signals into Several Sub-Ranges and Summation of these Sub-Ranges

1.1. Various strategies are available for dividing the signals into several sub-ranges. For example, it is possible to sort by retention time of the LC/MS signals or by mass and to summarise accordingly.

| | Range 1 | | | Range 2 | | | Range 3 | | | Range 4 | | |
|---|---|---|---|---|---|---|---|---|---|---|---|---|
| | Signal | Signal | Signal | Signal | Signal | Signal | Signal | Signal | Signal | Signal | Signal | Signal |
| | 1 | 2 | 3 | 4 | 5 | 6 | 7 | 8 | 9 | 10 | 11 | 12 |
| Batch_1 | 16711 | 5763 | 14366 | 8912 | 8521 | 24000 | 29252 | 11208 | 9471 | 8302 | 7189 | 5317 |
| Batch_2 | 16797 | 5961 | 14480 | 8713 | 9170 | 23294 | 25059 | 5834 | 7969 | 8250 | 5102 | 3937 |
| Batch_3 | 16411 | 6182 | 14636 | 8096 | 7357 | 23788 | 21161 | 7111 | 9148 | 7458 | 4075 | 5450 |
| Batch_4 | 16800 | 7030 | 12939 | 8800 | 8293 | 24433 | 19279 | 6319 | 9599 | 8005 | 4291 | 6829 |
| Batch_5 | 16677 | 6739 | 13440 | 9141 | 9954 | 23389 | 20415 | 6159 | 10361 | 8324 | 2171 | 6645 |

1.2. Summation of the signals from the sub-ranges, then continue with step iii)

| | Range 1 | Range 2 | Range 3 | Range 4 |
|---|---|---|---|---|
| Batch_1 | 36841 | 41433 | 49932 | 20808 |
| Batch_2 | 37238 | 41177 | 38862 | 17290 |
| Batch_3 | 37229 | 39241 | 37421 | 16983 |
| Batch_4 | 36769 | 41526 | 35197 | 19126 |
| Batch_5 | 36855 | 42485 | 36934 | 17140 |

2.1. Another possibility arises from the use of the mass defect plot, with the help of which the signals may be divided on the basis of their chemical substance class. The mass defect of each signal is calculated as follows:

$$MD = \frac{mz - floor ( mz )}{mz} * {10}^{6}$$

MD=Mass defect

mz=m/z ratio to 4 decimal places

floor( )=function that rounds a decimal number to the nearest integer

Example Calculation for Some m/z Ratios

| mz | | mz − floor (mz) | MD |
|---|---|---|---|
| (measured) | floor(mz) | mz | (Mass defect) |
| 183.1748 | 183 | 0.00095428 | 954.2797372 |
| 187.0981 | 187 | 0.000524324 | 524.3238707 |
| 357.0568 | 357 | 0.000159078 | 159.0783315 |
| 585.1575 | 585 | 0.000269158 | 269.1583035 |
| 839.1849 | 839 | 0.000220333 | 220.3328492 |

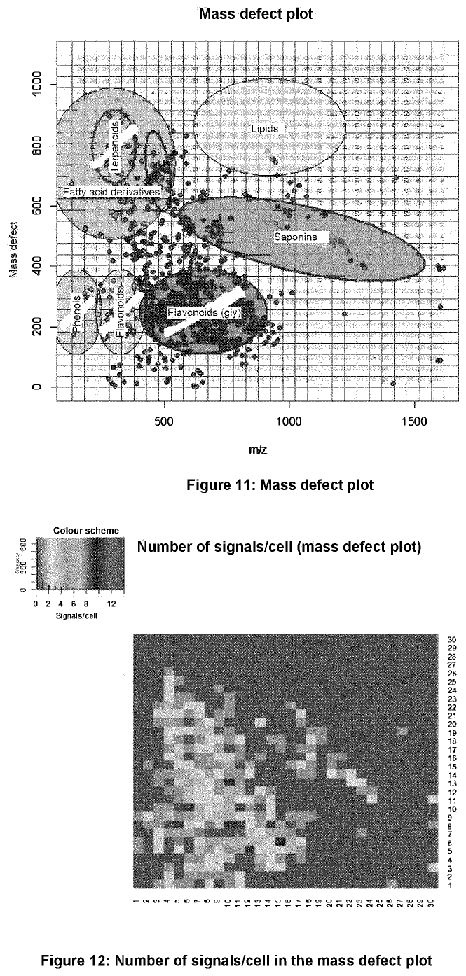

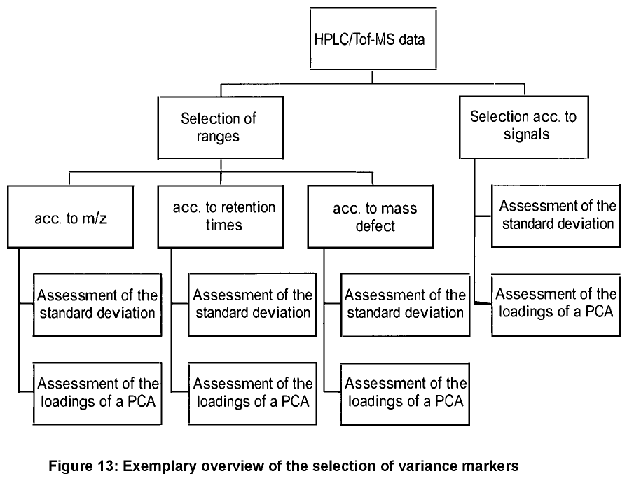

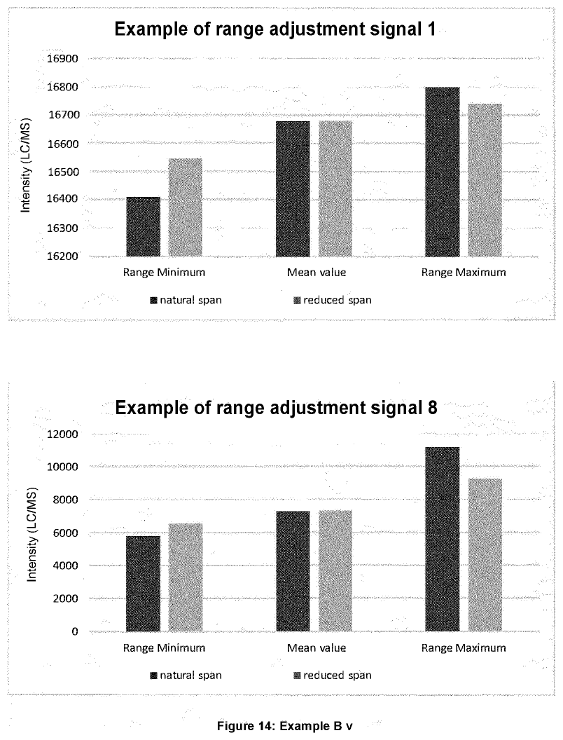

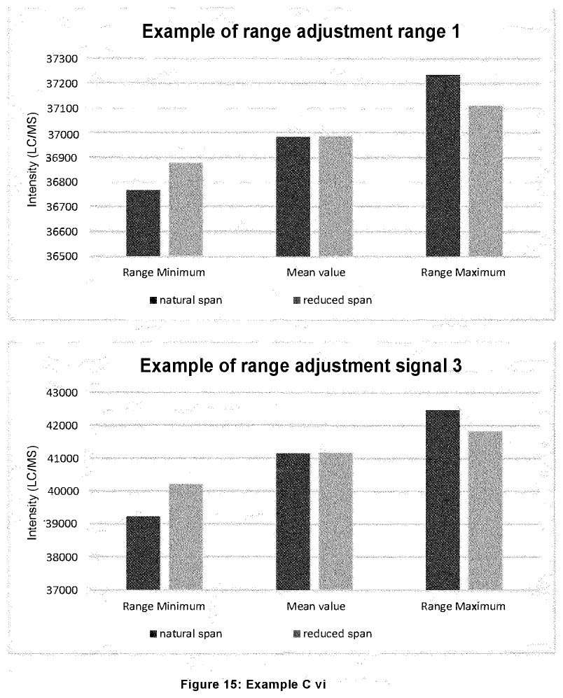

The result is a graph like in FIG. 11, in which each signal may be plotted in a coordinate system where the X-axis is the m/z-ratio and the Y-axis is the corresponding mass defect. The position of the signal is usually characteristic for the substance group (e.g. flavonoids, terpenoids, etc.) to which the signal belongs.

2.2. Then, for example, with the help of the grid superimposed on it (shown here in grey), the sum of the intensities of the signals contained in each cell may be calculated for each batch.

2.3. For overview purposes, the number of signals per cell (several signals of different intensity may be contained per cell) may be displayed in a heat map (see FIG. 12).

2.4. The summation of the signal intensities in the individual cells results, for example, in the following excerpt (cells whose sum is 0, because there is no signal there, have been excluded. The heading always follows the scheme “Cell (coordinate on X-axis|coordinate on Y-axis)”).

| | Cell | Cell | Cell | Cell | Cell |
|---|---|---|---|---|---|
| | (1\|5) | (1\|6) | (1\|9) | (2\|8) | (2\|9) |
| Batch 1 | 1430 | 4703 | 541 | 1671 | 4298 |
| Batch 2 | 2577 | 5058 | 950 | 2133 | 4306 |
| Batch 3 | 2664 | 4939 | 1046 | 2502 | 4309 |
| Batch 4 | 2808 | 5186 | 971 | 2526 | 4306 |
| Batch 5 | 1717 | 4316 | 600 | 1817 | 4300 |

With this table it is then possible to continue analogously from the following point iii); the rest of the procedure thereafter is identical.

iii) Identification of the Sub-Ranges that Contribute Most to the Variance/Standard Deviation

Calculation of the Loadings of the Table of Step 1.2 (or 2.4) by Means of a Principal Component Analysis (Here for Example 4 Principal Components; More Principal Components May be Selected at any Time):

| | PC1 | PC2 | PC3 | PC4 |
|---|---|---|---|---|
| Range 1 | 0.5560277 | −0.3779871 | 0.4745952 | −0.568083 |
| Range 2 | −0.4228279 | 0.588995 | 0.6291389 | −0.2801532 |
| Range 3 | −0.4177076 | −0.6292769 | 0.4980767 | 0.4259701 |
| Range 4 | −0.5810078 | −0.3379663 | −0.3617503 | −0.6460227 |

Summation of the Absolute Values for all Ranges:

| Range 1 | Range 2 | Range 3 | Range 4 |
|---|---|---|---|
| 1.976693 | 1.921115 | 1.9710313 | 1.9267471 |

Sorting from the Largest to the Smallest Value—Identification of the 2 Most Important Areas for Later Optimisation (Here Underlined; More Areas can Also be Selected for Later Optimisation):

| Range 1 | Range 3 | Range 4 | Range 2 |
|---|---|---|---|
| 1.976693 | 1.9710313 | 1.9267471 | 1.921115 |

iv) Determination of the Phytonutrient Content Underlying the Variance Markers

This may be performed optionally, so that specific contents (for example in mg/L or the like) are assigned to the intensities. For this purpose, corresponding quantification methods are available for various analytical methods. However, this step may also be skipped (as in this example), as this only represents a linear transformation of the intensities.

v) Determination of the Natural Span of the Selected Ranges

| | Range 1 | Range 3 |
|---|---|---|
| Range Minimum | 36769 | 35197 |
| Mean value | 36986 | 39669 |
| Range Maximum | 37238 | 49932 |

vi) Determination of a Reduced Span

The allowed minimum is raised, the allowed maximum is lowered, thus reducing the allowed span of the mixture(s) (see FIG. 15):

| | Range 1 | Range 3 |
|---|---|---|
| New permitted | 36878 | 37433 |
| minimum | | |
| Mean value | 36986 | 39669 |
| New permitted | 37112 | 44801 |
| maximum | | |

vii) Performance of a Mixing Calculation

After the Calculation, the Following Handling Instruction, for Example, Results for the Batches to be Used for Mixing:

| | Share in % |
|---|---|
| Batch 1 | 50.00 |
| Batch 2 | 20.00 |
| Batch 5 | 30.00 |

Expected Result for this Mixture Example:

| | Range 1 | Range 3 |
|---|---|---|
| Expected signal | 36924 | 43819 |
| intensities | | |

All expected signal intensities are within the desired reduced span.

viii) This Calculation May be Repeated Accordingly, for Example, Remixing Other Batches.

### Example D

Examples of how the LC/MS Signals that can be Used for Optimisation are Detected within the Scope of the Mixture Calculation

Below are described 2 possible examples:
a) By evaluating the principal component analysis (PCA) of an LC/MS measurement of a sample of crude drugs
b) By evaluating the standard deviations of measured LC/MS signals of a sample of crude drugs

For reasons of space, only excerpts of the original data sets are shown here to convey the principle.

With Regard to a):

i) Determination of Signals of Phytonutrients by LC/MS for Several Batches

The result is typically a table like the following—the numbers are the measured intensity values from, for example, an LC/MS measurement. (In the following example, 40 batches were measured and 363 signals were determined in each case; the following table is an excerpt).

| | Signal | Signal | Signal | Signal | Signal | Signal | Signal | Signal | Signal | Signal | Signal | Signal |
|---|---|---|---|---|---|---|---|---|---|---|---|---|
| | 1 | 2 | 3 | 4 | 5 | 6 | 7 | 8 | 9 | 10 | 11 | 12 |
| Batch_1 | 9418 | 20502 | 15661 | 8338 | 19523 | 5265 | 769 | 4071 | 3142 | 3710 | 53741 | 5275 |
| Batch_2 | 12067 | 24221 | 15079 | 3575 | 19903 | 5549 | 7052 | 3991 | 3373 | 10319 | 55016 | 5898 |
| Batch_3 | 10880 | 18391 | 15361 | 4120 | 19271 | 4581 | 5274 | 3660 | 2731 | 4308 | 52762 | 4763 |
| Batch_4 | 12972 | 19288 | 16197 | 7262 | 21719 | 7748 | 7889 | 4790 | 2897 | 6482 | 57076 | 7193 |
| Batch_5 | 11923 | 17665 | 17322 | 5598 | 21461 | 6120 | 7760 | 4734 | 3110 | 5847 | 56114 | 6207 |

ii) Carrying Out a Principal Component Analysis (PCA)

Presentation of Scores & Loadings

Of the loadings, in particular, 6 principal components are calculated and presented here—this corresponds to a total variance of around 80% in the present example and therefore describes the data set sufficiently well. However, it is always possible to include further principal components in order to increase the total variance explained.

| | PC1 | PC2 | PC3 | PC4 | PC5 | PC6 |
|---|---|---|---|---|---|---|
| Share variance (%) | 27.552 | 23.735 | 10.933 | 7.845 | 5.311 | 4.809 |
| Cumulative variance (%) | 27.552 | 51.287 | 62.22 | 70.065 | 75.376 | 80.185 |

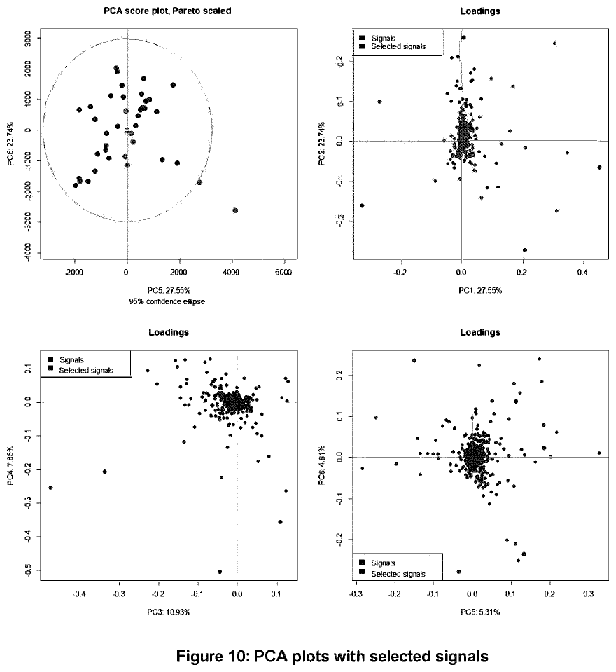

As described in example A, the absolute value is calculated from the loading values obtained, summed over the selected principal components, and then sorted by size in descending order. In FIG. 10, the 5 signals contributing most to the variance were selected and marked in red in the graphs of the loading plot (presentation of 2 of the 6 principal components per plot) (the selection of further signals is possible at any time). The signals marked in red are indeed always the same signals—only represented in the different principal components. They cover the complete span of the signals and represent the samples sufficiently well.

The signals selected in this way can then be used for the mixture calculation as described in Example A.

With Regard to b):

An alternative signal selection is possible by simply looking at the standard deviation of the signal intensities from batches measured by LC/MS. For example, the 5 signals with the highest standard deviation can be identified and also used for the mixture calculation.

i) Measurement of Several Batches by LC/MS (Here: Excerpt of Obtained Signal Table) and Calculation of Standard Deviations for Each Signal

| | Signal | Signal | Signal | Signal | Signal | Signal | Signal | Signal | Signal | Signal | Signal | Signal |
|---|---|---|---|---|---|---|---|---|---|---|---|---|
| | 1 | 2 | 3 | 4 | 5 | 6 | 7 | 8 | 9 | 10 | 11 | 12 |
| Batch_1 | 9418 | 20502 | 15661 | 8338 | 19523 | 5265 | 769 | 4071 | 3142 | 3710 | 53741 | 5275 |
| Batch_2 | 12067 | 24221 | 15079 | 3575 | 19903 | 5549 | 7052 | 3991 | 3373 | 10319 | 55016 | 5898 |
| Batch_3 | 10880 | 18391 | 15361 | 4120 | 19271 | 4581 | 5274 | 3660 | 2731 | 4308 | 52762 | 4763 |
| Batch_4 | 12972 | 19288 | 16197 | 7262 | 21719 | 7748 | 7889 | 4790 | 2897 | 6482 | 57076 | 7193 |
| Batch_5 | 11923 | 17665 | 17322 | 5598 | 21461 | 6120 | 7760 | 4734 | 3110 | 5847 | 56114 | 6207 |
| Standard | 1358 | 2580 | 884 | 2024 | 1135 | 1196 | 2973 | 493 | 246 | 2595 | 1741 | 928 |
| deviation | | | | | | | | | | | | |

ii) Sorting/Identifying the Signals with the Highest Standard Deviation

In this example, 5 signals have been selected (more are possible at any time) and underlined.

| | Standard deviation |
|---|---|
| Signal 7 | 2972.69048 |
| Signal 10 | 2594.5976 |
| Signal 2 | 2579.53471 |
| Signal 4 | 2024.25463 |
| Signal 11 | 1741.25076 |
| Signal 1 | 1357.8297 |
| Signal 6 | 1195.66479 |
| Signal 5 | 1135.03692 |
| Signal 12 | 927.916591 |
| Signal 3 | 884.462549 |
| Signal 8 | 493.222769 |
| Signal 9 | 245.744379 |

With these selected signals, a mixture calculation may also be performed, as already explained elsewhere. This second method identifies largely the same signals as the method via principal component analysis (PCA).

For example, if 20 instead of only 5 signals are selected using the two methods, the selection in this example still shows a match of 17 signals (see FIG. 9).

1.-10. (canceled)

11. A method for producing plant material having reduced variance in phytonutrient content, said method having the following steps:
i) determining signal intensities for phytonutrients in two or more batches by means of a detector, in particular GC-MS and/or LC-MS,
ii) identifying at least one phytonutrient which makes a contribution, preferably the greatest contribution, to the variance and identifying its determination of the natural span,
iii) setting one or more limit values which is/are smaller than the natural span from ii),
iv) mixing at least two batches, wherein at least one limit value from iii) is taken into consideration by means of a mixture calculator,
v) optionally, repeating steps i) to iv).

12. A method for producing plant material having reduced variance in phytonutrient content, said method having the following steps:
i) determining signal intensities for phytonutrients in two or more batches by means of a detector, in particular GC-MS and/or LC-MS,
ii) dividing the signals into two or more sub-ranges, and summing the signal intensities of the phytonutrients within each sub-range,
iii) identifying at least one sub-range which makes a contribution, preferably the greatest contribution, to the variance and identifying its determination of the natural span,
iv) setting one or more limit values which is/are smaller than the natural span from iii),
v) mixing at least two batches, wherein at least one limit value from iv) is taken into consideration by means of a mixture calculator,
vi) optionally, repeating steps i) to v).

13. The method for producing plant material having reduced variance in phytonutrient content according to claim 11, characterised in that at least one detector is selected from the group of liquid chromatography (LC), UV-VIS, heat conductivity detector, flame ionisation detector, or detectors that display signal intensities in a chromatogram in dependence on the retention time.

14. The method for producing plant material having reduced variance in phytonutrient content according to claim 11, characterised in that at least 100, 200 or 300 signal intensities or more are determined in a batch.

15. The method for producing plant material having reduced variance in phytonutrient content according to claim 11, characterised in that the signal intensities are determined in 5 or 10 batches.

16. The method for producing plant material having reduced variance in phytonutrient content according to claim 11, characterised in that the plant material is selected from the group consisting of Equiseti, Juglandis, Millefolii, Quercus, Taraxaci, Althaeae, Matricariae, Centaurium, Levisticum, Rosmarinus, Angelica, Artemisia, Astragalus, Leonurus, Salvia, Saposhnikovia, Scutellaria, Siegesbeckia, Armoracia, Capsicum, Cistus, Echinacea, Galphimia, Hedera, Melia, Olea, Pelargonium, Phytolacca, Primula, Salix, Thymus, Vitex, Vitis, Rumicis, Verbena, Sambucus, Gentiana, Cannabis, and Silybum.

17. The method for producing plant material having reduced variance in phytonutrient content according to claim 11, characterised in that the plant material is a plant extract.

18. The method for producing plant material having reduced variance in phytonutrient content according to claim 11, characterised in that a principal component analysis (PCA) is used.

19. A plant material having reduced variance in phytonutrient content obtained by the method according to claim 11.

20. A plant material having reduced variance in phytonutrient content or with reduced batch variability.

21. The method for producing plant material having reduced variance in phytonutrient content according to claim 12, characterised in that at least one detector is selected from the group of liquid chromatography (LC), UV-VIS, heat conductivity detector, flame ionisation detector, or detectors that display signal intensities in a chromatogram in dependence on the retention time.
